(19) United States
(12) Plant Patent Application Publication
(10) Pub. No.: US 2016/0360659 P1
(43) Pub. Date: Dec. 8, 2016

(54) Hop plant named 'HBC 567'

(50) ;

(71) Applicants: Eugene G. Probasco, Yakima, WA (US); Jason Perrault, Toppenish, WA (US)

(72) Inventors: Eugene G. Probasco, Yakima, WA (US); Jason Perrault, Toppenish, WA (US)

(21) Appl. No.: 14/756,962

(22) Filed: Nov. 2, 2015

Related U.S. Application Data
(60) Provisional application No. 62/230,412, filed on Jun. 4, 2015.

(51) Int. Cl. A01H 5/00

(52) U.S. Cl. PLT/236

(57) ABSTRACT

A new hop plant named ‘HBC 567’ is disclosed. The cones of ‘HBC 567’ mature in September. ‘HBC 567’ is used for its aromatic quality.

### CROSS-REFERENCE TO RELATED APPLICATIONS

Priority is claimed with respect to U.S. Provisional Patent Application Ser. No. 62/230,412 filed on Jun. 4, 2015.

### STATEMENT REGARDING FEDERALLY SPONSORED RESEARCH OR DEVELOPMENT

None

### GENUS AND SPECIES

Humulus lupulus

### VARIETY DENOMINATION

‘HBC 567’

### BACKGROUND OF THE INVENTION

‘HBC 567’ is a product of a controlled breeding program carried out by the inventors in the Yakima Valley of Washington State. Prior to 2014, the original single plant of ‘HBC 567’ was expanded to multiple plants, which were planted in the area of Toppenish, Wash. By 2014, the plants had been observed and evaluated for several years. Throughout several generations of asexual propagation, ‘HBC 567’ has been observed to retain its distinctive characteristics and remain true to type.

### BRIEF DESCRIPTION OF THE PHOTOGRAPHS

FIG. 1 illustrates a mature ‘HBC 567’ hop plant grown on a trellis;

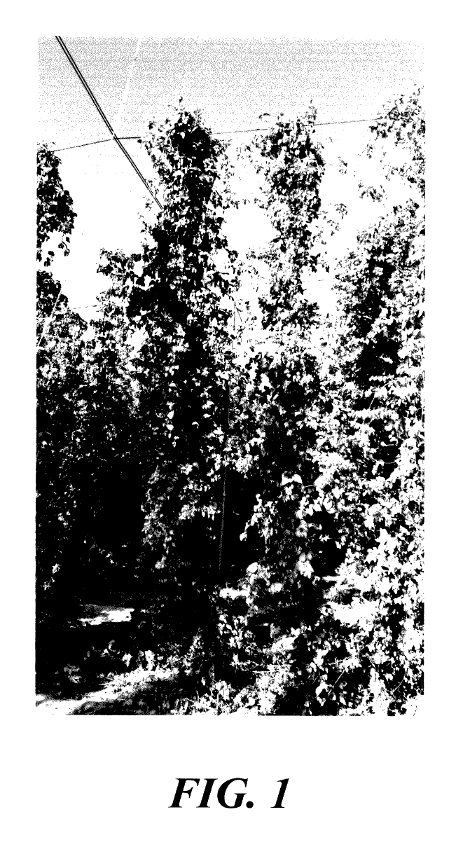

FIG. 2 illustrates whole cones and cross sections of cones of the ‘HBC 567’ hop plant;

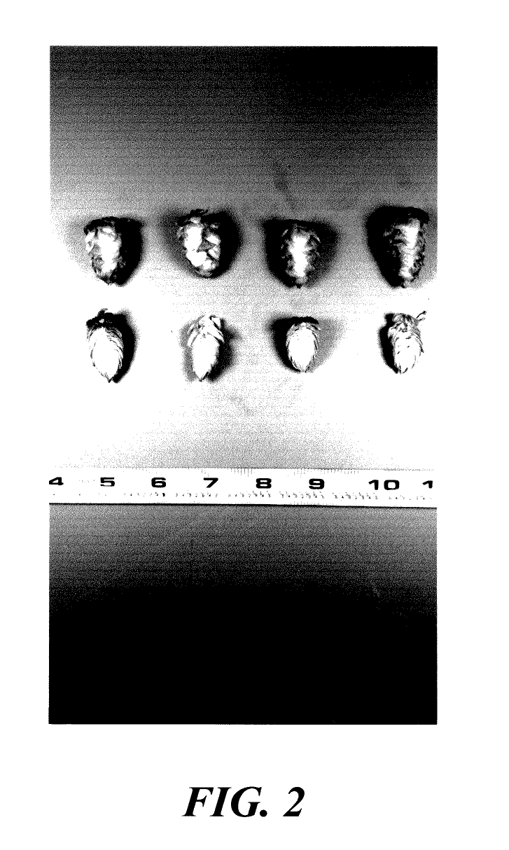

FIG. 3 illustrates the bine of a mature ‘HBC 567’ hop plant; and.

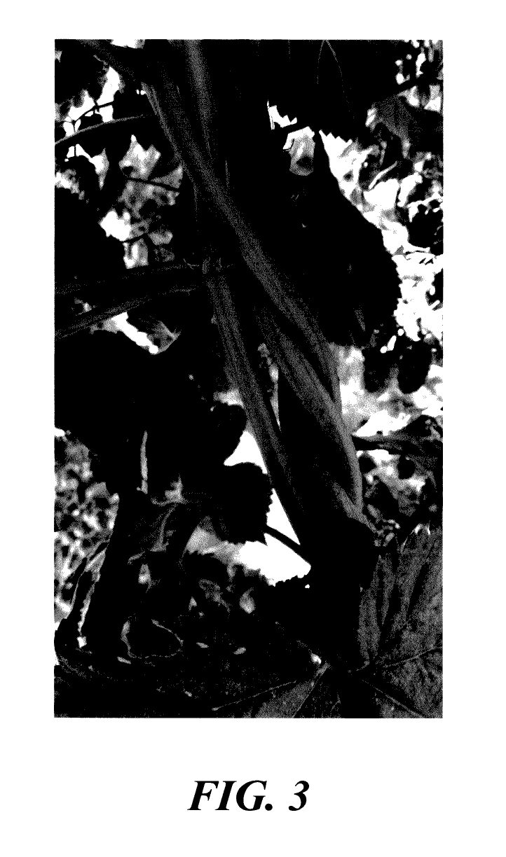

FIG. 4 illustrates the cones and leaves of a mature ‘HBC 567’ hop plant.

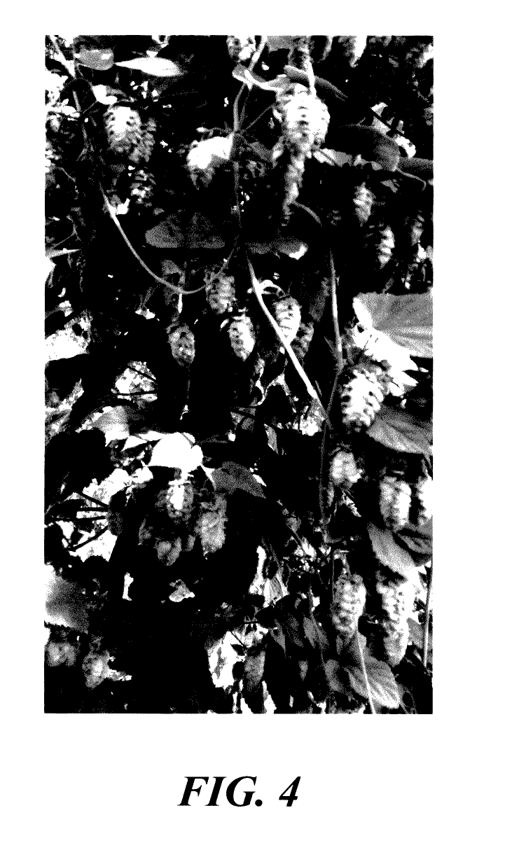

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2013-2014 growing seasons at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Use: Brewing
Harvest date: September (during 2013 to 2014 growing seasons at Toppenish, Wash.)
Bine:
   Color.—Yellow Green 146D.
   Stripe present.—Yes.
   Stripe color.—Red Purple 60B.
   Bine diameter.—13 mm at base and 16 mm at nine feet.
   Length between internodes.—7.5 cm.
   Lateral Length.—47.6.
   Lateral diameter.—4.5 mm at base and 1.4 at terminal end.
   Lateral color.—Yellow Green 146D.
Leaf:
   Color of mature leaf upper surface.—Yellow Green 147A.
   Color of mature leaf lower surface.—Yellow Green 147B.
   Number of lobes.—1-7.
   Margin.—Serrate.

1. What is claimed is a new and distinct hop plant as shown and described herein.
